(19) United States
(12) Plant Patent Application Publication
(10) Pub. No.: US 2015/0327424 P1
(43) Pub. Date: Nov. 12, 2015

(54) Dianella caerulea Plant Named 'DCGL'

(50) ;

(71) Applicant: Vic Ciccolella, Oakville (AU)

(72) Inventor: Vic Ciccolella, Oakville (AU)

(21) Appl. No.: 14/120,216

(22) Filed: May 6, 2014

(51) Int. Cl. A01H 5/00

(52) U.S. Cl. PLT/424

(57) ABSTRACT

‘DCGL’ is a distinctive variety of Dianella caerulea which is characterized by the presence of yellow and green leaf variegation and uniformity of traits through successive cycles of asexual propagation.

### LATIN NAME OF THE GENUS AND SPECIES

The Latin name of the genus and species of the novel variety disclosed herein is Dianella caerulea.

### VARIETY DENOMINATION

The inventive variety of Dianella caerulea disclosed herein has been given the variety denomination ‘DCGL’.

### BACKGROUND OF THE INVENTION

The present invention relates to a new and distinct perennial variety of Dianella caerulea, which has been given the variety denomination of ‘DCGL’. Its market class is that of an ornamental plant or grass-like plant. ‘DCGL’ is intended for use in landscaping and container gardening.

Parentage: In 2005, seed that resulted from an open pollination of a ‘no-cane’ form of Dianella caerulea (unnamed, unpatented) was sown at a nursery in Oakville, New South Wales, Australia and the resulting progeny were grown to a mature size. In February 2006, the selection now called ‘DCGL’ was observed to have green and yellow variegated foliage whereas the parent and other sibling progeny did not have variegated foliage; said selection was isolated for further observation. It was further grown, and subsequently observed from February 2006 until April 2013, at which time it was determined that the characteristics for which it was originally selected were uniform and stable.

Asexual Reproduction: In January 2007, ‘DCGL’ the selection was propagated by vegetative divisions and said divisions were grown to mature plants. These plants were subsequently divided in October 2007 to further increase numbers and test for stability. In 2011, ‘DCGL’ was initiated into tissue culture. It has been uniform and stable through all generations of division and subcultures. ‘DCGL’ was grown on between November 2011 and April 2013 and has shown that the characters for which it was selected are uniform and stable with no off types observed.

### SUMMARY OF THE INVENTION

‘DCGL’ is a distinctive variety of Dianella caerulea which is characterized by the presence of yellow and green leaf variegation and uniformity of traits through successive cycles of asexual propagation.

### BRIEF DESCRIPTION OF THE FIGURES

FIG. 1 illustrates an exemplary mature ‘DCGL’ plant.

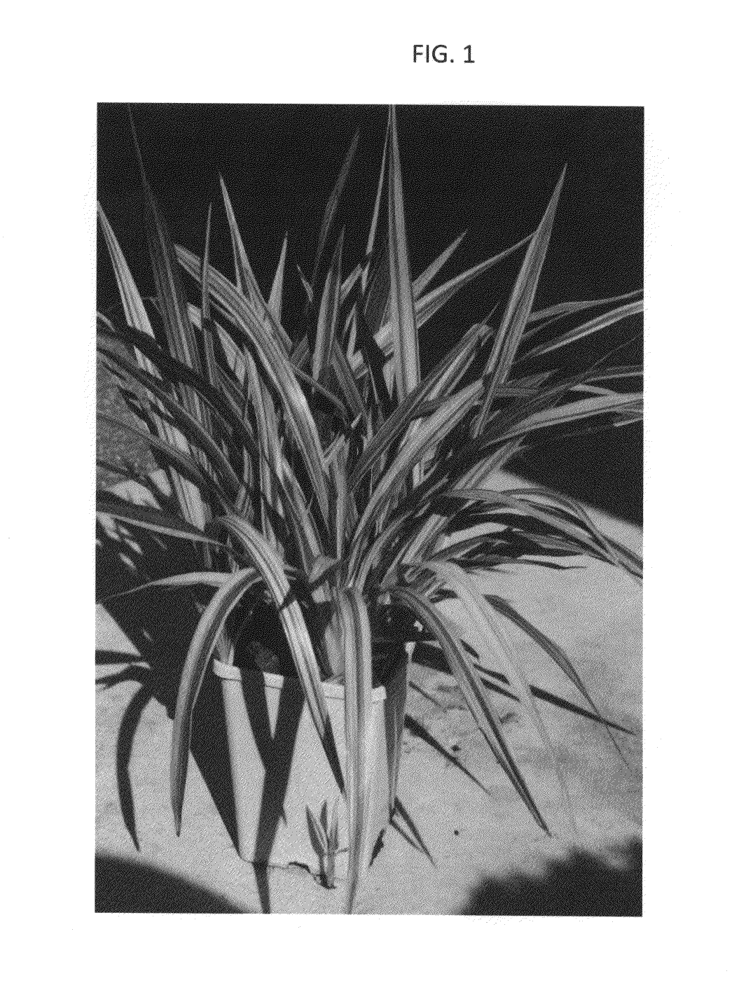

FIG. 2 illustrates the exemplary foliage variegation, the short rhizomes, and strong shoot density of ‘DCGL’.

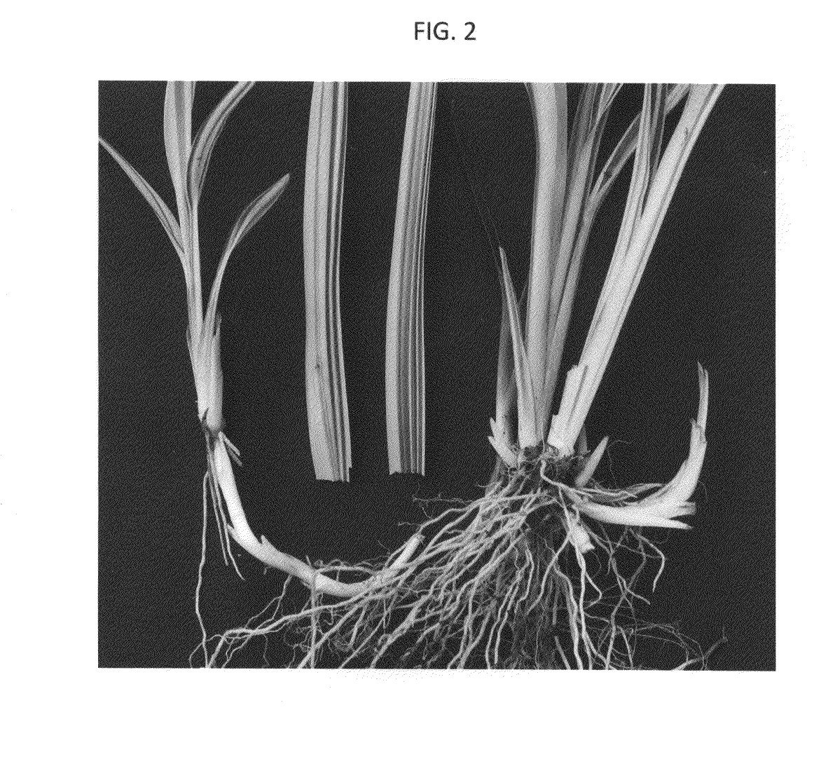

### BOTANICAL DESCRIPTION OF THE PLANT

The following is a detailed botanical description of a new and distinct variety of a Dianella caerulea ornamental plant known as ‘DCGL’. Plant observations were made on plants grown in New South Wales, Australia. Unless indicated otherwise, the descriptions disclosed herein are based upon observations made from mature ‘DCGL’ plants grown from rooted cuttings from May 2012 to May 2013 in 200 mm nursery pots filled with soilless potting media, maintained with granular slow release fertilizer, and regularly watered with overhead irrigation. No pest and disease measures were taken.

Those skilled in the art will appreciate that certain characteristics will vary with older or, conversely, younger plants. ‘DCGL’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Unless otherwise indicated, color notations are based on The Royal Horticultural Society Colour Chart, The Royal Horticultural Society, London, 2001 edition. Note that generic color descriptions such as ‘yellow’ do not exist in the R.H.S. charts and the corresponding R.H.S. colors are quoted.
Growth habit, dimensions and color:
Plant description:
   Plant habit.—Semi-erect, grass-like perennial.
   Height.—35 cm.
   Width.—30 cm.
   Bloom period.—Late spring.
   Hardiness.—USDA Zone 9 to 11.
   Environmental tolerances.—‘DCGL’ has not yet been observed under all conditions but has shown to be heat tolerant, adapting well to temperatures of 103 degrees Fahrenheit without any noticeable damage. It has survived light to moderate frosts and temperatures down to 23 degrees Fahrenheit.
   Drought tolerance.—‘VOC1’ has not yet been observed under all conditions but it has shown good drought tolerance once established; typical of the species.
   Pest and disease susceptibility or resistance.—In common with the species, none of note.
   Propagation.—Propagation is accomplished by dividing the rhizomatous crown of the plant.
   Time to produce a rooted cutting.—3 weeks at approximately 68 degrees Fahrenheit.
   Crop time.—From 4 to 7 months are needed to produce a well-rooted 200 mm pot, starting from a rooted cutting, depending on geographic location.
Stem:
   Branching habit.—Acaulescent, rhizomatous plant with shoots emerging upright at 90 degrees from rhizomes.
Roots: Short, subsurface rhizomes which root at nodes; roots fibrous; root density is high.
Basal shoots:
   Shoots density.—numerous; more than 25 in a 200 mm nursery pot.
   Shoot strength.—Strong.
   Cross section.—Equitant.
   Shoot color.—Cream, closest to 160D.
   Shoot dimensions.—Newly emerging shoots with mean length of 21 mm and a mean diameter of 4.4 mm.
   Shoot surface.—Smooth.
Foliage:
   Type.—Evergreen.
   Arrangement.—Equitant.
   Division.—Simple.
   Shape.—Linear.
   Apex.—Acute.
   Base.—Sheathed.
   Venation.—Parallel.
   Vein color (adaxial surfaces).—same as surrounding foliage.
   Vein color (abaxial surfaces).—same as surrounding foliage.
   Margins.—Serrulate.
   Attachment.—Sessile.
   Texture.—Smooth.
   Surfaces (adaxial surface).—Glabrous.
   Surfaces (abaxial surface).—Glabrous.
   Mature leaf dimensions.—average length 35 cm, average width 2.1 cm.
   Leaf color (adaxial & abaxial surfaces).—Juvenile and mature foliage is variegated with approximately fifty percent of the leaf surface being green, corresponding closest to RHS 146A, and fifty percent being yellow, corresponding to RHS 12A.
   Petiole.—Leaves are sessile.
   Stipules.—Absent.
Inflorescence: Lax panicles sitting above the foliage appear in late spring; number of flowers per inflorescence is more than 30 in a mature 200 mm nursery pot.
Flowers: Not yet observed.
Reproductive organs: Not yet observed.
Fruit and seed production: Not yet observed.
Comparison of DCGL with Other Varieties of Dianella caerulea While there are several variegated forms of the Genus known to the breeder, ‘DCGL’ is the only variegated form of this species, making the parent plant the closest available comparator. ‘DCGL’ is very similar to the parent plant, a ‘no-cane’ form of Dianella caerulea (unnamed, unpatented), in most respects except for leaf color. ‘DGCL’ has a yellow and green leaf variegation corresponding to RHS 12A and RHS 146A, respectively, whereas the parent plant has solid green foliage, corresponding to 146A.

That which is claimed is:

1. A new and distinct variety of Dianella caerulea plant named ‘DCGL’, substantially as described and illustrated herein.
